(19) United States
(12) Patent Application Publication
(10) Pub. No.: US 2025/0319042 A1
(43) Pub. Date: Oct. 16, 2025

(54) SKIN CARE PRODUCT WITH LAPACHOL AND METHOD FOR USE THEREOF

(71) Applicant: Steve Salzinger, Scarsdale, NY (US)

(72) Inventor: Steve Salzinger, Scarsdale, NY (US)

(21) Appl. No.: 19/061,207

(22) Filed: Feb. 24, 2025

Related U.S. Application Data
(60) Provisional application No. 63/557,461, filed on Feb. 23, 2024.

(51) Int. Cl. A61K 31/122; A61K 8/31; A61K 8/34; A61K 8/35; A61K 8/37; A61K 8/81; A61K 8/92; A61P 17/00; A61Q 19/00; A61Q 19/08

(52) U.S. Cl. CPC A61K 31/122; A61K 8/31; A61K 8/342; A61K 8/345; A61K 8/355; A61K 8/375; A61K 8/8188; A61K 8/922; A61P 17/00; A61Q 19/00; A61Q 19/08

(57) ABSTRACT

A method is provided to treat a subject in need of treatment for erythema, including facial erythema from any one of acne, rosacea, photoaging, heat/burns, heat from laser treatments, and/or injury scars that are in a red stage. The method includes topically administering to the subject in need thereof a composition having a therapeutically effective amount of lapachol and one or more acceptable excipients. The therapeutically effective amount correlates to the composition having approximately to 0.25% of lapachol by volume, and the administration of the composition being an application to an area affected by erythema twice per day.

### RELATED APPLICATION

This application claims the benefit of priority from U.S. Provisional Patent Application No. 63/557,461, filed on Feb. 23, 2024, the entirety of which is incorporated by reference.

### FIELD OF THE INVENTION

The present arrangement relates to a skin care product including lapachol. More particularly, the present arrangement relates to a skin care product including lapachol and a related method for treating redness/erythema.

### BACKGROUND

Facial erythema is a common dermatologic problem caused by acne, rosacea, or photoaging that is challenging to treat. Topical anti-inflammatory antibiotics, such as minocycline and metronidazole, have been used to address facial redness associated with both acne and rosacea. Other treatments involve the use of vasoconstrictors, such as oxymetazoline and brimonidine. While this approach effectively reduces facial erythema in acne, rosacea, and photoaging, the improvement is largely cosmetic since the facial erythema returns when the vasoconstrictor effect is no longer present. Cosmetic formulations for facial redness reduction have examined the use of topical botanical anti-inflammatories, such as allantoin and bisabolol. Allantoin is found in plants, such as comfrey, horse chestnut, and bearberry, but it is usually synthesized for cosmetic formulations from urea. Allantoin forms the basis for many sensitive skin formulations but is not sufficient for noticeable redness reduction. Bisabolol, a chamomile extract, is also a botanical anti-inflammatory sometimes combined with allantoin, or other botanical extracts also used for sensitive skin and redness reduction. While allantoin and bisabolol are some of the most effective over the counter (OTC) ingredients for redness reduction, the list of substances touted for redness reduction in the cosmetic realm is endless to include niacinamide, Centella asiatica, aloe vera, azelaic acid, ceramides, squalene, tea tree, echinacea, lavender, etc.

### OBJECTS AND SUMMERY

One ingredient that is available over the counter (OTC) is lapachol (2-hydroxy-3-(3-methyl-2-butenyl)-1,4-naphtoquinone), also known as pau d'arco. It was identified in 1882 and is derived from the inner bark of the Tabebuia tree native to Central and South America. The tree grows up to one hundred and twenty-five (125) feet tall and is rich in the naphthoquinone lapachol. It is used both topically and orally as an anti-inflammatory, but has also been shown to exhibit antibacterial, antifungal, and anticancer activities. The bark extract inhibits the growth of methicillin-resistant Staphylococcus aureus (MRSA), Helicobacter pylori, Enerococcus, Clostridium, Candida albicans, and Cryptococcus neoformans by interring with the electron transport chasing inhibiting cellular respiration. It is thought to produce its anti-inflammatory and antinociceptive effects by inhibiting the release of histamine. In addition, beta-lapachone, a compound related to lapachol, inhibits prostaglandin E2 (PGE2), tumor necrosis factor alpha (TNF-a), matrix metalloproteinase 3 (MMP-3), MMP-8, and MMP-9 in cell culture. The inclusion of lapachol may have been shown in some instances to improve the appearance of scars and skin damage by supporting the skin's natural healing process.

However, the present arrangement provides for a lapachol-containing moisturizer that reduces erythema, including facial erythema from acne, rosacea, photoaging, heat/burns, heat from laser treatments, and/or injury scars that are in a red stage.

### DETAILED DESCRIPTION

In at least one embodiment, a lapachol-containing moisturizer is provided for reducing erythema, including facial erythema, may include, but is not limited to, ingredients from the following:
Water/Aqua >50%
Triethyl Citrate 5%-10%
Helianthus annuus (Sunflower) Seed Oil 5%-10%
Butyrospermum Parkii (Shea) Butter 5%-10%
Caprylic/Capric Triglyceride 1%-5%
Glycerin 1%-5%
Squalane 1%-5%
Behenyl Alcohol 1%-5%
Isopentyldiol 1%-5%
Ammonium Acryloyldimethyltaurate/VP Copolymer 1%-5%
Benzyl Alcohol 0.1%-1%
Oryza sativa (Rice) Bran Extract 0.1-1%
Stearyl Alcohol 0.1-1%
Glyceryl Stearate 0.1-1%
Xanthan Gum 0.1-1%
Phytosterol 0.1-1%
Helianthus annuus (Sunflower) Extract 0.1-1%
Sclerotium Gum 0.1-1%
Lapachol 0.1-1%
Hydrogenated Lecithin 0.1-1%
Sodium Hyaluronate 0.1-1%
Polyglyceryl-10 Myristate 0.1-1%
Lecithin <0.1%
Rosmarinus officinalis (Rosemary) Leaf Extract <0.1%
Pullulan <0.1%
Tocopherol <0.1%
Dehydroacetic Acid <0.1%
Hydroxypropyl Cyclodextrin <0.1%
Palmitoyl Tripeptide-38<0.1%
Sodium Benzoate <0.1%
Acetyl Hexapeptide-8<0.1%

It is noted that the listed percentages are percentages by weight for the moisturizer. The listed percentages are considered exemplary, and modifications thereof may still fall within the scope of the present arrangement.

In the preferred embodiment, the inclusion of lapachol may be included in the overall formulation at 0.25% by volume, however, it is contemplated that the amount may be increased to up to 1% by volume.

In order to test such formulation a trial was conducted using twenty-five (25) healthy female and male subjects 35-65 years of age of Fitzpatrick skin types I-II with mild to moderate stable facial erythema from acne, rosacea, or photoaging. Subjects signed an informed consent prior to any study activities (Allendale Institutional Review Board (AIRB), Old Lyme, CT). Subjects had to answer “yes” to the question “Do you have more red spots on your face than twelve (12) months ago?” Subjects were asked to continue their own, self-selected cleanser, sunscreen, cosmetics, and other skin care products throughout the eight (8)-week study they had used for the prior thirty (30) days without difficulty.

Subjects received the study treatment product for twice daily morning and evening application using an exemplary formulation including an amount of lapachol at 0.25% as well as ingredients selected from the list above.

Subjects were assessed at baseline, week four (4), and week eight (8). The dermatologist investigator and subjects assessed efficacy and tolerability at each visit on a five (5)-point ordinal scale (0=none, 1=minimal, 2=mild, 3=moderate, 4=severe). Visia CR4.3 (Canfield Scientific, Piscataway, New Jersey) images were taken of all subjects at baseline, week four (4), and week eight (8) using standard lighting one (1) and cross polarized light of the central, right, and left face. Noninvasive erythema assessments of the face were conducted using a colorimeter (Minolta, Japan) and dermaspectrophotometer (Cortex Technologies, Hansund, Denmark) at baseline, week four (4), and week eight (8).

Investigator and subject ordinal nonparametric data were analyzed using Wilcoxon signed rank test with comparison to baseline. The noninvasive parametric data was analyzed using paired t-test. Change was considered significant at the alpha level of 0.05.

The results of the test on the twenty-five (25) subjects are as follows:

Twenty five of the twenty-five (25/25) subjects successfully completed the clinical trial without any adverse events or adverse experiences. The study enrolled twenty-one (21) females and four (4) males including twelve (12) subjects with rosacea, six (6) subjects with photoaging, and seven (7) subjects with acne were enrolled. The study enrolled twenty-one (21) females (F) and four (4) males (M). Twenty-four (24) of the subjects were Caucasian and one (1) subject was Hispanic. No tolerability issues were noted by the investigator in terms of worsening dryness, peeling, erythema, and edema or the subjects in terms of worsening dryness, peeling, stinging, or itching.

Facial erythema was assessed with two noninvasive techniques: colorimeter and dermaspectrophotometer. The colorimeter, which assesses the absorbance and transmittance of light in the visible spectrum, assessed facial color in three-point color space defined as L*a*b* (L* indicates light/dark, a* represents red/green, b* represents blue/yellow). There was a 4% increase at week four (4) and a 5% increase at week eight (8) on the L* axis indicating more skin brightness, sometimes terms radiance in the skin care industry. On the a* scale, there was a 26% decrease in skin redness at week four (4) and a 31% decrease in skin redness at week eight (8). The b* value on the blue/yellow scale did not have meaning for this research.

A dermaspectrophotometer was used as a second device utilizing different optic technology to assess skin erythema. The dermaspectrophotometer utilizes two light emitting diodes: one emitting green light in the 568 nm range and the second emitting red light in the 655 nm range. These diodes illuminate the skin surface and record the intensity of red light using a photodetector. After four (4) weeks of use, a 20% reduction in erythema was noted and after eight (8) weeks a 29% reduction in erythema was observed. Thus, the DSP erythema scale data was consistent with the colorimeter data.

The dermatologist investigator assessed efficacy in terms of facial erythema, fine lines, wrinkles, radiance/brightness, skin roughness (tactile), skin roughness (visual), and overall appearance at baseline, week four (4), week eight (8). At week eight (8), there was a 44% improvement in erythema, a 37% improvement in radiance, a 34% improvement in tactile roughness, a 37% reduction in visual roughness, and a 25% improvement in overall facial skin appearance. No statistically significant improvement was seen in fine lines or wrinkles by the investigator. The subjects assessed efficacy in terms of facial erythema, fine lines, wrinkles, radiance/brightness, skin roughness (tactile), skin roughness (visual), and overall appearance at baseline, week four (4), week eight (8). By week eight (8), the subjects noted a 40% improvement in redness, a 17% improvement in lines, a 14% improvement in wrinkles, a 39% improvement in radiance, 35% improvement in tactile roughness, a 37% improvement in visual roughness, and a 39% improvement in overall skin appearance. The subject and investigator assessments were consistent with representative before and after images.

It is noted that facial erythema reduction in the cosmetic realm requires combining ingredients with different mechanisms of action to achieve a cumulative beneficial effect. Lapachol at approximately 0.25% was the primary active ingredient in the facial moisturizer. The facial moisturizer also has a formulation that utilizes a variety of ingredients selected from the list above while achieving the observed clinical results. Of primary importance in formulations for facial erythema is a vehicle that restores the skin barrier to minimize noxious sensory stimuli and penetration of unwanted substances.

The study formulation was based on, in at least one embodiment, lapachol, combined with a variety of botanical oils, to provide an occlusive layer over the skin surface and reduce evaporative water loss, humectants, to assist in the water holding capacity of the formulation, emollients to impart skin smoothness and softness, peptides to reinforce the dermal/epidermal junction, and anti-inflammatories to botanically supplement the lapachol.

For example, in at least some embodiments of the moisturizer product, such ingredients listed above may be used, in addition to lapachol, for their properties as listed below:
Water/Aqua—Solvent
Triethyl Citrate—Emollient
Helianthus annuus (Sunflower) Seed Oil—Emollient
Butyrospermum Parkii (Shea) Butter—Emollient
Caprylic/Capric Triglyceride—Emollient
Glycerin—Humectant
Squalane—Occlusive Agent
Behenyl Alcohol—Co-Emulsifier
Isopentyldiol—Humectant
Ammonium Acryloyldimethyltaurate/VP Copolymer—Gellant
Benzyl Alcohol—Preservative
Oryza sativa (Rice) Bran Extract—Conditioning Agent
Stearyl Alcohol—Co-Emulsifier
Glyceryl Stearate—Emulsifier
Xanthan Gum—Emulsion Stabilizer
Phytosterol—Co-Emulsifier
Helianthus annuus (Sunflower) Extract—Emollient
Sclerotium Gum—Stabilizer
Hydrogenated Lecithin—Co-emulsifier
Sodium Hyaluronate—Conditioning Agent
Polyglyceryl-10 Myristate—Emulsifier
Lecithin—Co-Emulsifier
Rosmarinus officinalis (Rosemary) Leaf Extract—Antioxidant
Pullulan—Stabilizer
Tocopherol—Antioxidant
Dehydroacetic Acid—Preservative
Hydroxypropyl Cyclodextrin—Conditioning Agent
Palmitoyl Tripeptide-38—Conditioning Agent
Sodium Benzoate—Preservative
Acetyl Hexapeptide-8—Conditioning Agent

It is noted that lapachol is primarily oil-soluble, the ingredients listed below collectively act as a solvent system in dissolving the lapachol to help it retain its ability to stay stable, even with a primarily water based solvent system:
Triethyl Citrate,
Helianthus annuus (Sunflower) Seed Oil,
Butyrospermum Parkii (Shea) Butter,
Caprylic/Capric Triglyceride,
Glycerin,
Squalane,
Behenyl Alcohol, Oryza sativa (Rice) Bran Extract,
Stearyl Alcohol,
Glyceryl Stearate,
Phytosterols,
Hydrogenated Lecithin,
Polyglyceryl-10 Myristate,
Lecithin, Rosmarinus officinalis (Rosemary) Leaf Extract,
Pullulan,
Tocopherol

In one embodiment, the process for formulating the moisturizer follows a emulsion system where such oil soluble ingredients are combined, including the lapachol, and melted together then added to the water phase to form the emulsion, along with the remainder of the ingredients.

The study formulation discussed above addressed facial redness by restoring the skin barrier, occluding further water loss, enhancing skin water holding capacity, and providing a cocktail of anti-inflammatory compounds while improving skin tactile and visual aesthetics. These improved skin qualities were observed by the investigator and subject assessments with confirmation from two methods of optical facial erythema assessment. Topical facial application of a lapachol-containing moisturizer was found to be effective in reducing facial erythema resulting from acne, rosacea, and photoaging.

While only certain features of the invention have been illustrated and described herein, many modifications, substitutions, changes or equivalents will now occur to those skilled in the art. It is therefore to be understood that this application is intended to cover all such modifications and changes that fall within the true spirit of the invention.

1. A method of treating a subject in need of treatment for erythema, including facial erythema from any one of acne, rosacea, photoaging, heat/burns, heat from laser treatments, and/or injury scars that are in a red stage, said method comprising the steps of:
topically administering to the subject in need thereof a composition consisting essentially of a therapeutically effective amount of lapachol and one or more acceptable excipients,
wherein the therapeutically effective amount correlates to said composition having approximately to 0.25% of lapachol by volume, and the administration of said composition being an application to an area affected by erythema twice per day.

2. The method as claimed in claim 1, wherein said composition is water based.

3. The method as claimed in claim 1, wherein said one or more acceptable excipients is selected from the group consisting of: Water/Aqua, Triethyl Citrate, Helianthus annuus (Sunflower) Seed Oil, Butyrospermum Parkii (Shea) Butter, Caprylic/Capric Triglyceride, Glycerin, Squalane, Behenyl Alcohol, Isopentyldiol, Ammonium Acryloyldimethyltaurate/VP Copolymer, Benzyl Alcohol, Oryza sativa (Rice) Bran Extract, Stearyl Alcohol, Glyceryl Stearate, Xanthan Gum, Phytosterol, Helianthus annuus (Sunflower) Extract, Sclerotium Gum, Hydrogenated Lecithin, Sodium Hyaluronate, Polyglyceryl-10 Myristate, Lecithin, Rosmarinus officinalis (Rosemary) Leaf Extract, Pullulan, Tocopherol, Dehydroacetic Acid, Hydroxypropyl Cyclodextrin, Palmitoyl Tripeptide-38, Sodium Benzoate, Acetyl Hexapeptide-8.

4. The method as claimed in claim 3, wherein if said one or more acceptable excipients is Triethyl Citrate, it is included in said composition at approximately 5-10% by volume.

5. The method as claimed in claim 3, wherein if said one or more acceptable excipients is Helianthus annuus (Sunflower) Seed Oil, it is included in said composition at approximately 5-10% by volume.

6. The method as claimed in claim 3, wherein if said one or more acceptable excipients is Butyrospermum Parkii (Shea) Butter, it is included in said composition at approximately 5-10% by volume.

7. The method as claimed in claim 3, wherein if said one or more acceptable excipients is Caprylic/Capric Triglyceride, it is included in said composition at approximately 1-5% by volume.

8. The method as claimed in claim 3, wherein if said one or more acceptable excipients is Glycerin, it is included in said composition at approximately 1-5% by volume.

9. The method as claimed in claim 3, wherein if said one or more acceptable excipients is Squalane, it is included in said composition at approximately 1-5% by volume.

10. The method as claimed in claim 3, wherein if said one or more acceptable excipients is Behenyl Alcohol, it is included in said composition at approximately 1-5% by volume.

11. The method as claimed in claim 2, wherein if said one or more acceptable excipients is Isopentyldiol, it is included in said composition at approximately 1-5% by volume.

12. The method as claimed in claim 2, wherein if said one or more acceptable excipients is Ammonium Acryloyldimethyltaurate/VP Copolymer, it is included in said composition at approximately 1-5% by volume.

13. The method as claimed in claim 1, wherein the therapeutically effective amount, including the administration of said composition is an application to said area affected by erythema twice per day, for four weeks.

14. The method as claimed in claim 1, wherein the therapeutically effective amount, including the administration of said composition is an application to said area affected by erythema twice per day, for eight weeks.

15. The method as claimed in claim 1, wherein said topical administration of said composition consisting essentially of (a) a therapeutically effective amount of lapachol and (b) one or more acceptable excipients, is sufficient to generate any one of: a 44% improvement in erythema, a 37% improvement in radiance, a 34% improvement in tactile roughness, a 37% reduction in visual roughness, and/or a 25% improvement in overall facial skin appearance, as assessed by a dermatologist investigator, after an application to said area affected by erythema twice per day, for eight weeks.

16. The method as claimed in claim 1, wherein said topical administration of said composition consisting essentially of (a) a therapeutically effective amount of lapachol and (b) one or more acceptable excipients, is sufficient to generate any one of: a 40% improvement in redness, a 17% improvement in lines, a 14% improvement in wrinkles, a 39% improvement in radiance, 35% improvement in tactile roughness, a 37% improvement in visual roughness, and/or a 39% improvement in overall skin appearance, as assessed by said subject, after an application to said area affected by erythema twice per day, for eight weeks.
